(19) United States
(12) Patent Application Publication
(10) Pub. No.: US 2009/0270643 A1
(43) Pub. Date: Oct. 29, 2009

(54) Method for producing an oxygen-containing compound used as fuel additive, in particular in diesel fuels, gasoline, and rapeseed methyl ester

(76) Inventors: Michiel Arjaan Kousemaker, Gasselternijveen (NL); Klaus Dieter Thiele, Hildesheim (DE)

Correspondence Address: COLLARD & ROE, P.C., 1077 NORTHERN BOULEVARD, ROSLYN, NY, 11576, US

(21) Appl. No.: 10/562,409

(22) PCT Filed: May 13, 2004
(86) PCT No.: PCT/DE04/00999

§ 371 Date: Sep. 8, 2006

(30) Foreign Application Priority Data
Jun. 24, 2003 (DE) 103 28 159.2

(51) Int. Cl. C07C 51/43

(52) U.S. Cl. 554/174

(57) ABSTRACT

The invention concerns a method for producing an oxygen-containing compound used as fuel additive, in particular in diesel fuels, gasoline and rapeseed methyl ester. The method consists in a) reacting a polyvalent alcohol with an aldehyde or a ketone to produce an acetal and b) etherifying the subsisting free hydroxyl groups in the acetal produced at step a) with tertiary olefins.

### OBJECT OF THE INVENTION

The invention describes the use and production of an oxygen-containing compound in Diesel fuels, gasoline, and rapeseed methyl ester, with which the readiness for ignition is increased and the particle emission is reduced. These improvements are achieved, for example, by a) production of 2,2 dimethyl-4-hydroxymethyl-1,3 dioxolan from glycerine and acetone and b) reacting of the 2,2 dimethyl-4-hydroxymethyl-1,3 dioxolan produced in step a) with isobutane in order to etherify the remaining OR group.

### DESCRIPTION OF THE PRIOR ART

The addition of oxygen compounds to fuels in the form of alcohols and ethers has proved its value. As a result of such use, it has proved possible to do away with the ecologically questionable lead compounds which served as anti-knock means in fuels. The problem therefore arose of producing a compound by the derivation of the glycerine molecule which is suitable as a fuel component.

The following compound classes are suitable compound classes which have in part already been publicised and are subject to protective rights:
Glycerine ether
Glycerine ester
Glycerine acetals
Glycerine ether

The production of glycerine ethers has been protected in a number of patent specifications. For example, the patent specification U.S. Pat. No. 1,968,033 describes the etherification of multivalent alcohols. The reacting of multivalent alcohols and tertiary olefins is dealt with in terms of protective rights under DE 4 222 183. A method variant for the large-scale technical production of glycerine ethers is developed in EP 649 829.

In addition to this general description for the production of glycerine ethers, special catalyst systems for use in this product class have been examined in various patent specifications. In DE 1 224 294 acidic fixed-bed catalysts are used for the reaction.

Glycerine ethers occur as a by-product in the separation of tertiary olefins from the C₄ fraction in the distillation of crude oil. This is dealt with in Patent Specification U.S. Pat. No. 1,968,601.

In Patent Specification U.S. Pat. No. 4,605,787 the production of alkyl tertiary alkyl ethers is described, whereby acidic zeolites are used as catalyst systems. The glycerine ethers are also used as phase agents in the reaction of glycerine and isobutene in DE 1 224 294.

In WO 94/01389 the production of polyalkyl ethers from polyhydroxy compounds with a high molecular weight is described.

Not only have the individual method steps for the production of glycerine ethers been dealt with in terms of patent law, but also their use as fuel components in Diesel fuels and gasoline.

It is known that the addition of oxygen-containing compounds in fuels results in an improvement in quality.

In WO 81/00721 a fuel mixture is described which has been modified by the addition of alcohols, water, ethers and vegetable oil. The Patent Specification U.S. Pat. No. 4,353,710 is also concerned with the modification of Diesel fuels with ethers and esters.

The addition of ethers to Diesel fuels has been described in DE 3 140 382.

The improvement of Diesel fuel quality by the addition of aliphatic polyethers has been proven in the Patent Specification U.S. Pat. No. 2,655,440. A mixture of alcohol and oxygenated hydrocarbons with a molecular weight from 250-500 was used in Patent Specification U.S. Pat. No. 4,753,661 for improving the quality of gasoline and Diesel fuel.

The invention described in U.S. Pat. No. 5,308,365 relates to a Diesel quality with low sulphur content by the addition of dialkyl and trialkyl derivatives of glycerine.

The use of these glycerine ethers is intended to serve to eliminate hydrophilicity, to incur a reduction in the boiling temperature in the range of the boiling diagram of the fuel components, and to achieve a reduction in density while maintaining the cetane number.

The disadvantage with these substances is that a glycerine-ether mixture is formed with a maximum of 11% of triethers. The remainder consists of monoethers and diethers, which because of the hydroxyl groups still present are in part not soluble in the individual fuel components.

Due to the steric hindrance, 100% conversion to triethers is not possible. The conversion reaction to glycerine ethers is almost thermoneutral and heavily entropic. This leads to the situation that, as the temperature increases, the yield is reduced and oligomerisation sets in. With a reduction in the reaction temperature, however, the reaction is slowed to such an extent that no reaction worth mentioning takes place.

### Glycerine Esters

The production of glycerine esters is described in the GDR Patent Specification 156 803. This relates to the production of triacetin.

The esterisation to lower glycerine esters does indeed bring the boiling point into the range of the Diesel fuel, but with a longer acryl reside no adequate ignition behaviour is achieved. On the other hand, the boiling behaviour of the triacetin is too high and therefore excludes use in gasoline. With glycerine esters, which lie within the boiling range of the conventional fuel components, solubility in the fuels is no longer guaranteed.

The disadvantage of this substance class lies in the inadequate physical characteristics which exclude use in gasoline fuels, and the deficient ignition readiness in Diesel fuels.

### Glycerine Acetals

The production of glycerine acetates is described in the publications by
R. R. Tink, E. Y. Speneer, J. M. Roxburg—Cann. J. Techn. 29 243 (1951) and
R. R. Tink; A. C. Neish—Can. J. Techn 29 243 (1951) using the example of the reaction of glycerine with butyraldehyde.

Dioxalans with longer alkyl residues, the production of which is described by C. Diantadosi (J. Org. Chem. 80;6613 (1958)) do not come into consideration for economic reasons.

Determinant for admixture in Diesel fuel, gasoline, and rapeseed methyl ester is its solubility in these fuel components. This is very problematic due to the hydroxyl group which is present, however Even if the boiling position of the glycerine in its acetals is drastically reduced, the density in all cases is perceptibly above 1.02 g/ml.

The use of these acetals in Diesel fuel was disappointing due to its poor ignition behaviour.

### BRIEF DESCRIPTION OF THE INVENTION

Glycerine, as an extremely hydrophilic substance, cannot be mixed either with gasoline (OK) or with Diesel fuel (DK) and rapeseed methyl ester (RME). The problem is to derivatise glycerine in such a way that the products can be used as fuel components in DK, OK, and rapeseed methyl ester. To do this, it is necessary for it to be made compatible with fuels in order to fulfil the fuel standards.

Compatibility with DK, OK, and RME is achieved by a complete reaction of the hydroxyl groups present at the glycerine molecule, on the one hand by the conversion of acetal and the etherification of the hydroxyl group still present by means of a tertiary olefin.

The derivatives produced in this way can be mixed in any proportion with DK, OK, and RME.

The addition of such substances resulted in a lower particle emission and a better ignition readiness than pure DK, OK, or RME.

### Example of Production

In a first reaction step, for example, glycerine is reacted with acetone to 2,2-dimethyl-4-hydroxymethyl-1,3 dioxalan. Next, the 2,2-dimethyl-4-hydroxymethyl-1,3 dioxalan is etherified in an acidic catalyst reaction with isobutene.

### Production of 2,2-dimethyl-4-hydroxymethyl-1,3 Dioxalan

In a 5-litre flask 600 g of glycerine (free of water), 3600 g acetone, and 2,5 g p-toluol sulphonic acid are mixed under powerful stirring. The reaction mixture is stirred at room temperature and then mixed with 60 g potassium carbonate (free of water). After further stirring for about 1 hour the reaction mixture is filtrated and the filtrate distilled in fractioned form.

After a forerun of acetone, which can be used in a further reaction, distillation takes place at a pressure of 15 Torr of the 2,2-dimethyl-4-hydroxymethyl-1,3 dioxalan in a boiling range from 82-84° C.

The yield is 550-600 g. According to gas chromatic testing, the substance contained a purity of >98% and a refraction index of n=1,432.

### Production of 2,2-dimethyl-4-hydroxymethyl-1,3 Dioxalan Tert Butyl Ether

300 g of the 2,2-dimethyl-4-hydroxymethyl-1,3 dioxalan is presented in an autoclave with 2.5 g p-toluol sulphonic acid and cooled to −30° C. 600 g isobutene is then added. This mixture is brought to reaction with a magnetic stirrer at 90° C.

After a relatively small forerun, the end product moves at 20 Torr into a boiling interval of 82-85° C.

The reactions described are repeated several times over and produce on average yields of 300 g.

According to gas chromatographic testing, with the reaction described a purity of >95% was obtained and a refraction index n_{D}, dependent on the purity, of 1.4190-1.4260.

In order to obtain a purity of >99%, phenyl isocyanate was added to the distillate and heated under backflow. Fractioning then again took place in a vacuum. The desired product in this case was obtained, according to gas chromatographic testing, in a purity of >99%.

### The Use of 2,2-dimethyl-4-hydroxymethyl-1,3 Dioxalan Tert Butyl Ether (STBE) as a Fuel Component

The substance described above was mixed as an additive to Diesel fuels, gasolines, and rapeseed ethyl esters. In this situation we determined that the particle emission is reduced in the form of clouding and the readiness to ignite increased in the form of dp max.

| | Clouding [%] | dp max bar/° kW |
|---|---|---|
| Conventional commercial | | |
| Diesel fuel | 2.20 | 6.68 |
| Diesel fuel + 20% STBE | 1.16 | 8.13 |
| RME | 1.03 | 5.61 |
| RME + 20% STBE | 0.00 | 6.43 |

1. Method for producing an oxygen-containing compound used as fuel additive, in particular in Diesel fuels, gasoline, and rapeseed methyl ester, comprising
a): Reaction of a multivalent alcohol with an aldehyde or ketone to produce an acetal, and
b): Etherification of the still free hydroxyl groups of the acetal produced in step a) with tertiary olefins.

2. Method according to claim 1, wherein the multivalent alcohol in step a) is selected from the group which comprises trivalent to hexavalent alcohols, in particular triols such as glycerine, tetrols, pentols, trimethylolpropane, penta erythrite and sugar alcohols with 4 to 6 hydroxyl groups.

3. Method according to claim 1, wherein the aldehyde, the dialdehyde, or the ketone in step a) contains three to seven carbon atoms, whereby preferably acetaldehyde, acetone, or butyral aldehyde can be used.

4. Method according to claim 1, wherein the tertiary olefin in step b) is selected from the group which comprises i-butene, 2-methyl-1-butene, 2-methyl-2-butene, isomer hexene with a tertiary carbon atom at the double bond, isomer heptene with a tertiary carbon atom at the double bond, and hydrocarbon mixtures which contain i-butene, such as in raffinate 1 of the crude oil distillation, and for preference C₄ and/or C₅ tert. alkenes.

5. Method according to claim 1, wherein the raw materials for producing the oxygen-containing compound are selected in such a way that the oxygen-containing compound produced dissolves completely in the fuel in particular in Diesel fuel, gasoline, and/or rapeseed methyl ester.

6. Method according to claim 1, wherein the raw materials for producing the oxygen-containing compound are selected in such a way that the addition of the oxygen-containing compound produced to the fuel, in particular to Diesel fuel, gasoline, and/or rapeseed methyl ester, does not exert a negative influence on the flash point of the fuel, in particular of the Diesel fuel, gasoline, and/or rapeseed methyl ester.

7. Method according to claim 1, wherein the raw materials for producing the oxygen-containing compound are selected in such a way that the addition of the oxygen-containing compound produced to the fuel, in particular to Diesel fuel, gasoline, and/or rapeseed methyl ester, does not increase the water solubility of the fuel, in particular of Diesel fuel, gasoline, and/or rapeseed methyl ester.

8. An oxygen-containing compound produced according to a the method according to claim 1.

9. The oxygen-containing compound according to claim 8 produced by:
a): Reaction of a multivalent alcohol, preferably glycerine, with an aldehyde or ketone to produce an acetal, and
b): Etherification of the still free hydroxyl groups of the acetal produced in step a) with tertiary olefins to a complete reaction of the hydroxyl groups,
which oxygen-containing compound is completely soluable in the fuel, in particular the Diesel fuel, gasoline, and/or rapeseed methy ester.

10. The oxygen-containing compound according to claim 8, wherein the oxygen-containing compound has a purity of more than 95%.

11. The oxygen-containing compound according to claim 8, wherein the ketone is acetone and the tertiary olefin is i-butene.

12. The oxygen-containing compound according to claim 11, wherein the multivalent alcohol is glycerine to form the oxygen-containing compound 2,2-Dimethyl-4-hydroxymethyl-1,3 dioxolan-tert butylether.

13. The use of the oxygen-containing compound according to claim 8 as an additive for fuels, in particular Diesel fuels, gasolines, and rapeseed methyl esters, in quantities from 0.1% by volume to maximum 30% by volume.

14. Fuels, in particular Diesel fuels, gasolines, or rapeseed methyl esters, comprising 0.1% by volume to maximum 30% by volume of a completely dissolved oxygen-containing compound according to claim 8.
